(19) United States
(12) Patent Application Publication
(10) Pub. No.: US 2008/0020126 A1
(43) Pub. Date: Jan. 24, 2008

(54) Compound for and method of developing latent fingerprints

(75) Inventor: Douglas C. Arndt, Ventura, CA (US)

Correspondence Address: Harold L. Jackson;Jackson Law Corporation, Ste. N, 14751 Plaza Dr., Tustin, CA, 92780, US

(73) Assignee: Armor Holdings Forensics LLC

(21) Appl. No.: 11/804,193

(22) Filed: May 17, 2007

Related U.S. Application Data
(60) Provisional application No. 60/832,203, filed on Jul. 20, 2006.
(60) Provisional application No. 60/801,311, filed on May 18, 2006.

(51) Int. Cl. A61B 5/117

(52) U.S. Cl. 427/1; 252/301.35

(57) ABSTRACT

A latent fingerprint developing compound is formed by mixing cyanoacrylate, a solvent, and a fluorogenic reagent to provide a homogenous blend. The mixture is then solidified with the mixture having the characteristic of being capable of sublimating at a given temperature and in its sublimated state reacting with one or more of the constituent components of the residue of a latent fingerprint to form a discernable fluorescent image of the print. A latent fingerprint may be developed by exposing a substrate containing the latent print to the sublimated fumes of the compound.

### RELATED APPLICATION

This application claims priority of U.S. Provisional Application Ser. No. 60/832,203 (“'203 application”) entitled Compound for and Method of Developing Latent Fingerprints, filed Jul. 20, 2006 and of U.S. Provisional Application Ser. No. 60/801,311 ('311 application) entitled Compound for and Method of Developing Latent Fingerprints, filed May 18, 2006 for all common subject matter. The contents of the '203 and '311 applications are incorporated herein by reference.

### FIELD OF THE INVENTION

The present invention relates to the development of latent fingerprints.

### BACKGROUND OF THE INVENTION

The term “chemical developer” as used in the forensic fingerprint art refers to the visualization of the residue components, e.g., oils, salts, amino acids, deposited by the ridge pattern of a person's fingertip, i.e., fingerprint area, onto a porous substrate such as paper. It is to be noted that the term “fingerprint” or “fingerprint area” as used herein includes palm as well as foot prints.

Several methods have been used to develop latent fingerprints particularly on porous substrates such as paper. One method, described in my U.S. Pat. No. 6,841,188 (“'188 patent”), involves the preparation of a reagent solution, i.e., a complexing agent 8-hydroxyquinoline or derivative by mixing it with a metal salt and applying the solution, e.g., by spraying, dipping etc. to a porous substrate wherein the solution is adsorbed and precipates onto the substrate surface allowing the precipate to highlight the latent image.

While this method has achieved considerable success it has certain disadvantages including (a) to some extent shelf life limitations, (b) possible staining or blackening of the underlying documents, (c) possible destruction of the latent print residues and (d) the requirement that the substrate be porous.

The '188 patent discusses another prior art latent print developing method which involves the reduction of a silver salt in solution to elemental silver which precipates and adsorbs onto the surface of the latent print. The disadvantages of this method are also discussed in the '188 patent.

Iodine and ruthenium tetroxide have also been used in a fuming process as well as vacuum sublimation of relatively high vapor pressure chemicals and dyes. Ninhydrin and DFO solutions have also been used to develop latent prints. However, such solutions require that the materials be mixed and weighed. In addition, such solutions are generally flammable, provide inhalation hazards, often cause inks to run, and they can take several hours to weeks to develop prints. One company markets a refrigerant to which the ninhydrin or DFO can be added to overcome many of the problems, but it is quite expensive.

In addition to the above prior art latent fingerprint developing methods, cyanoacrylates, commonly referred to as Super Glue® in vapor form, has been used for this purpose. Cyanoacrylate (sometimes hereinafter referred to as “CA”) in liquid form consists of monomers of cyanoacrylate molecules (C5H5N02) an acrylic resin which rapidly polymerizes in the presence of water.

Fumes of cyanoacrylates have been used to render latent fingerprints more visible. Typical cyanoacrylates used for this purpose are the methyl and ethyl esters of cyanoacrylic acid, although other esters exist. By means of a process known as chlorosis, which involves the use of chlorine and water and may include the use of alkali hydroxides as a catalyst fumes of cyanoacrylate are directed onto substrate surfaces likely to have latent fingerprints thereon. The fumes polymerize to form a milky to white deposit following the fingerprint ridge pattern. Fingerprints developed in this manner are more visible against the background of the substrate surface where they are found and they may become permanently fixed to the surface as an insoluble preservation of the prints. It is often desirable to enhance the visibility of CA processed prints because they are commonly found on surfaces that produce little contrast, or on multicolored or on textured surfaces that can tend to obscure the fingerprints. CA fumes may be generated using a number of thermal methods. Liquid CA, which is not yet polymerized and is still in its monomeric state, is readily vaporized using heat sources such as a coffee warmer (electrical hot plate) or by exothermic chemical reactions involving sodium hydroxide, water, and balls of cotton, or involving calcium oxide and water. Unpolymerized CA can also be vaporized using a vacuum chamber without a heat source, although thermal assistance increases the efficiency. Polymerized CA, which is solid, is more difficult to vaporize and generally involves the need for a higher temperature. Laboratory hot plates may be necessary to produce the fumes from polymerized CA. Alternatively, portable butane torches having a quantity of CA contained in a replaceable nozzle are popular as means to fume polymerized CA. Enhancement of CA processed prints is presently performed using one or more fluorescent dyes in solution applied to the print subsequent to the CA fuming step.

Shortcomings of this enhancement procedure include:

1. The expenses associated with processing time, necessary chemicals and lab equipment;

2. Post treatment as an extra procedure;

3. The inconvenience of rinsing evidence (i.e., the latent print) with water to remove excess stain;

4. Restricting the staining process to labs rather than field use;

5. Obscuring the prints due to staining of the background surface;

6. Solvent destruction of the substrate or surface thereof;

7. Occupational and environmental hazards associated with flammable solvents;

8. The limited shelf life of dye solutions through chemical degradation; and

9. The need for hazardous waste disposal.

Attempts have been made to stain latent fingerprints as a component of the CA fuming process. These attempts have generally failed to achieve both viable dye staining and development with CA. The most successful attempt that I have been made aware of only produced fluorescence lasting a few seconds and this temporary presence of the fluorescent prints apparently prevented it from becoming commercially viable. Those skilled in the art are reported to have experimented with basic dyes, such as Basic Yellow-40, for example, subsequent to the CA fuming step. It is understood by the inventor of the present invention that this dye does not sublimate when heated at atmospheric pressures and that when it is heated, smoke is generated indicating it is undergoing thermal decomposition.

My earlier U.S. application Ser. No. 11/493,245 filed Jul. 26, 2006 (“'245 application”) based on an earlier provisional application, now published as US2007-0026130A1, describes a novel and relatively simple method of developing latent fingerprints in which the substrate containing the latent print is exposed to the gaseous phase of a chemical developer compound (normally in its solid phase) which reacts with one or more of the chemical residue constituents, e.g., oil, amino acids, etc. in a latent fingerprint. A list of satisfactory developer compounds are described in the '245 application as well as a preferred apparatus for containing and sublimating the compound in the presence of the substrate such as a document suspected of bearing the latent print. The contents of the '245 application are included herein by reference. The '245 method is geared to a closed environment, e.g., a compartment in which a planar substrate such as paper, cards, sheets, CD's, etc., is placed in close proximity to the chemical developer compound being heated.

A primary aspect of the present invention is to improve upon the '245 method by simultaneously developing latent fingerprints using a vaporized mixture of CA and a fluorogenic reagent. The present method is not limited to developing prints on planar objects such as paper sheets, but is applicable to the development of prints on 3-dimensional objects such as guns, beer bottles, knives, plastic bags containing drugs often found at crime scenes, or even the crime scenes themselves (rooms, the inside of automobiles, etc.) Another aspect of this invention is to target one or more specific fingerprint residue components, such as oils, salts and/or amino acids, and to simultaneously develop the targeted component(s) using a mixture of CA and a particular fluorogenic compound. In addition, it is an object of the present invention to provide a new fingerprint developer compound in the form of a mixture of CA and a fluorogenic reagent which can be sublimated, by heat for example, to expose the substrate purportedly bearing the latent print to the fumed mixture.

### SUMMARY OF THE INVENTION

A latent fingerprint developing method, in accordance with the present invention, includes (a) the provision of a fluorogenic reagent such as one of the reagents described in the '245 application which is compatible with CA and can sublimate before reaching its decomposition temperature, (b) mixing CA with the fluorogenic reagent and a suitable solvent to provide a homogenous CA fluorogenic reagent mixture, and (c) exposing the substrate or a portion thereof bearing the latent print to the CA fluorogenic reagent mixture in a gaseous phase, and (d) exposing the resultant print to radiation which may include radiation within and/or beyond the visible spectrum. Preferably the CA reagent mixture, prior to exposure to the latent print bearing substrate, is in its solid phase with heat being applied to vaporize some of the mixture to thereby expose the latent print to fumes of the CA fluorogenic reagent mixture.

With respect to a method of manufacturing my novel developer compound, I initially provide CA in liquid form, i.e., nonpolymerized form, and mix the liquid with a solvent containing the desired fluorogenic reagent to form a homogenous blend of the two ingredients. Then, I pour the liquid CA fluorogenic reagent mixture into one or more dispensers such as small metal cups, e.g, circular aluminum cups about ½″-2″ in diameter and ⅛″-⅜″ high and allow the mixture to solidify. Preferably, the solvent will provide a “pot life”, i.e., time before the mixture reaches a viscous state, which is appropriate for the chosen manufacturing process, e.g., 5-10 or more minutes. The latent fingerprint developer compound may then be used in the above method by heating one of the cups, in the presence of the substrate believed to bear a latent print, by means of a conventional small heat source such as a hot plate, butane torch, etc. The CA will form a white colored print which the fluorescent reagent will cause to fluoresce. The temperature required to sublimate the CA fluorogenic reagent mixture will depend upon the surrounding vapor pressure. The use of a vacuum chamber will lower the required fuming temperature. The required temperature to cause the CA fluorogenic reagent to sublimate is well within the knowledge of those skilled in the art.

### DESCRIPTION OF THE PREFERRED EMBODIMENTS

As is taught in the '245 application various fluorogenic reagents (or compounds) may be sublimated using heat to react with targeted groups of chemicals within latent fingerprint residues. These groups include sebaceous oils, amino acids, and salts. Some of the reagents or compounds disclosed in the '245 application will develop more than one group of residue chemicals, especially those which are polyfunctional reagents. Here is a partial list of fluorescent reagents suitable for the present invention along with the targeted constituent components of a latent fingerprint, i.e., oils, amino acids, etc.:

4-(dimethylamino)cinnamaldehyde-targets amino acids, but also reactive with salts

8-hydroxyquinoline-targets salts, but also reactive with amino acids

Nile Red, Coumarin 1, MBD (7-mehtoxybenzylamino-4-nitrobenzoxadiazole), carbazole, rhodamines,—targets oils

Dansyl chloride, fluorescamine—target amino acids

2-(2-hydroxyphenyl)benzoxazole and other low molecular weight benzoxazoles—non-specific Coumarin 152-non-specific

In addition the following reagents listed in the '245 application will be effective to a varying degree:

acridine and its derivatives, rhodamine dyes, 2-(2-hydroxyphenyl)-benzoxazole, 2-(2-hydroxyphenyl)benzothiazole, coumarin compounds such as 4-hydroxycoumarin and coumarins 1, 2,4,6,7,30,102,120,138,151,152,153,307,314,334,337,338, and 343, benzotriazole, 2-chloro-mercaptobenzoxazole, 2-phenylbenzothiazole, 2-phenylbenzaxazole, 8-hydroxyquinoline, 8-hydroxyquinaldine, anthracene and its derivatives, naphthalene and its derivatives, 4-(dimethylamino)cinnamaldehyde, fluorescamine, phthalic dicarboxaldehyde, naphthoquinone-4-sulfonic acid, dansyl chloride and other dansylated compounds, 4-chloro-7-nitrobenzofurazan, 4-dimethyaminobenzaldehyde, 5,6-dimethylbenzimidazole, 5-chloro-2-methylbenzothiazole, chrysene, 4-hydroxybenzaldehyde, nicotinamide, and camphor;

anthranilic acid, 1,4-naphthoquinone, benzanthrone, tetracene, pentacene, 2-(2-hydroxy-4-methylphenyl)-4-(3)-quinazolone, 2-(3,5-dichloro-2-hydroxyphenyl)-4-(3)-quinazolone, 2-(5-chloro-2-hydroxyphenyl)-4-(3)-quinazolone, 2-(2-hydroxy-3-methylphenyl)-4-(3)-quinazolone, 2-(4-ethyl-2-hydroxyphenyl)-4-(3)-quinazolone, anthraquinone, 1,4-benzoquinone, and salicylic acid; and

thiazoles, liposomes (e.g. Nile Red), stilbenes, azos, azolines, and conjugated polycyclic aromatic compounds containing at least three fused rings that include without limitation anthracene, benzanthracene, pentacene, substituted pentacene, naphthacene, phenacene, substituted phenacene, and derivatives thereof.

The preferred reagent is 2-2(hydroxyphenl)benzothiazole. It is relatively inexpensive. It is highly fluorescent in long wave, ultraviolet light, without a viewing filter. It is soluble in CA. It is best to first dissolve the fluorescent reagent in a solvent that is compatible with CA in terms of solubility, miscibility, and reactivity. In regards to reactivity, certain solvents (e.g., dimethyl formamide and dimethyl sulfoxide will cause CA to polymerize so rapidly as to make them impractical for production methods requiring a workable “pot-life.” A partial list of co-solvents includes ethyl lactate, ethyl acetate, acetone, gamma-butyrolactone, epsilon-caprolactone, 2-phenoxyethanol, carbitols, cellosolves, dichlorobenzene, ethyl benzene, methylene chloride, toluene, o-zylenes, and m-pyrrol. Co-solvents make it easy to obtain an homogenous blend. Some solvents will evaporate from the mixture while others will remain as part of the polymerized matrix. After the solvent(s), fluorophor(s), and CA re mixed in appropriate ratios, they may be poured into dispensers suitable for thermal vaporization. Appropriate ratios of materials are determined by the mixtures ability to:

1. Produce visual CA development of latent prints;

2. Produce a high level of fluorescence within the latent prints;

3. Not obscure the latent prints with background fluorescence; and

4. Product high resolution development of microscopic details (known in the art as “Level 3” details.

The use of a co-solvent which is compatible with CA and the fluorogenic regent is an important feature of this invention.

### EXAMPLE OF THE INVENTION

### Example 1

Equal parts by weight of ethyl cyanoacrylate and a 2% solution of 2-(2-hydroxyphenyl)benzothiazole in ethyl lactate.

### Example 2

Equal parts by weight ethyl cyanoacrylate and a saturated solution of Spectrafluor CO Yellow 6 (a roumarin dye-stuff distributed by Spectra Colors Corp.) in ethyl lactate. This mixture performs especially well to process latent prints found on polyethylene bags.

There has been described a novel and highly useful method of developing latent fingerprints. Various modifications are possible within the generic principles disclosed by this invention without departing from the spirit and scope of the present invention as defined in the appended claims.

What is claimed is:

1. A method of making a latent fingerprint developer compound comprising:
a) providing cyanoacrylate (CA) in liquid form;
b) providing a fluorogenic reagent which is soluble in the CA and which will sublimate a given temperature, the reagent being capable of reacting with constituent components found in the residue of a latent fingerprint to provide a discernable fluorescent image of the print;
c) mixing the CA and the fluorogenic reagent with a solvent to form a homogenous blend of the CA and reagent in solution;
d) pouring the mixture while in a liquid form into one or more dispensers; and
e) allowing the mixture to solidify.

2. The method of claim 1 wherein the fluorogenic reagent is one or more of the following reagents:
acridine and its derivatives, rhodamine dyes, 2-(2-hydroxyphenyl)-benzoxazole, 2-(2-hydroxyphenyl)benzothiazole, coumarin compounds such as 4-hydroxycoumarin and coumarins 1,2,4,6,7,30,102,120,138,151,152,153,307,314,334,337,338, and 343, benzotriazole, 2-chloro-mercaptobenzoxazole, 2-phenylbenzothiazole, 2-phenylbenzaxazole, 8-hydroxyquinoline, 8-hydroxyquinaldine, anthracene and its derivatives, naphthalene and its derivatives, 4-(dimethylamino)cinnamaldehyde, fluorescamine, phthalic dicarboxaldehyde, naphthoquinone-4-sulfonic acid, dansyl chloride and other dansylated compounds, 4-chloro-7-nitrobenzofurazan, 4-dimethyaminobenzaldehyde, 5,6-dimethylbenzimidazole, 5-chloro-2-methylbenzothiazole, chrysene, 4-hydroxybenzaldehyde, nicotinamide, and camphor;
anthranilic acid, 1,4-naphthoquinone, benzanthrone, tetracene, pentacene, 2-(2-hydroxy-4-methylphenyl)-4-(3)-quinazolone, 2-(3,5-dichloro-2-hydroxyphenyl)-4-(3)-quinazolone, 2-(5-chloro-2-hydroxyphenyl)-4-(3)-quinazolone, 2-(2-hydroxy-3-methylphenyl)-4-(3)-quinazolone, 2-(4-ethyl-2-hydroxyphenyl)-4-(3)-quinazolone, anthraquinone, 1,4-benzoquinone, and salicylic acid; and
thiazoles, liposomes (e.g. Nile Red), stilbenes, azos, azolines, and conjugated polycyclic aromatic compounds containing at least three fused rings that include without limitation anthracene, benzanthracene, pentacene, substituted pentacene, naphthacene, phenacene, substituted phenacene, and derivatives thereof.

3. The method of claim 1 wherein the fluorogenic reagent is one or more of the following reagents:
4-(dimethylamino)cinnamaldehyde,
8-hydroxyquinoline,
Nile Red, Coumarin 1, MBD (7-mehtoxybenzylamino-4-nitrobenzoxadiazole), carbazole, rhodamines,
Dansyl chloride, fluorescamine,
2-(2-hydroxyphenyl)benzoxazole and other low molecular weight benzoxazoles—non-specific Coumarin,
2-)2-hydroxyphenyl)benzothiazole and other low molecular weight benzothiazoles.

4. The method of claim 1 wherein the fluorogenic reagent is 2-(2-hydroxyphenyl)benzothiazole.

5. The method of claim 1 wherein the fluorogenic reagent is Spectrafluor CO Yellow 6 (a roumarin dye-stuff distributed by Spectra Colors Corp.).

6. The method of claim 1 wherein the solvent is selected from one or more of the following solvents:
ehtyl lactate, ethyl acetate, acetone, gamma-butyrolactone, epsilon-caprolactone, 2-phenoxyethanol, carbitols, cellosolves, dichlorobenzene, ethyl benzene, methylene chloride, toluene, o-zylenes, and m-pyrrol.

7. A latent fingerprint developer comprising a homogenous mixture of CA and a fluorogenic reagent in solid form, the mixture having the characteristic of being capable of sublimating at a given temperature and in its sublimated state reacting with one or more constituent components of the residue of a latent fingerprint to provide a discernable fluorescent image of the print.

8. The developer compound of claim 7 wherein the fluorogenic reagent is selected from one or more of the following reagents:
acridine and its derivatives, rhodamine dyes, 2-(2-hydroxyphenyl)-benzoxazole, 2-(2-hydroxyphenyl)benzothiazole, coumarin compounds such as 4-hydroxycoumarin and coumarins 1,2,4,6,7,30,102,120,138,151,152,153,307,314, 334,337,338, and 343, benzotriazole, 2-chloro-mercaptobenzoxazole, 2-phenylbenzothiazole, 2-phenylbenzaxazole, 8-hydroxyquinoline, 8-hydroxyquinaldine, anthracene and its derivatives, naphthalene and its derivatives, 4-(dimethylamino)cinnamaldehyde, fluorescamine, phthalic dicarboxaldehyde, naphthoquinone-4-sulfonic acid, dansyl chloride and other dansylated compounds, 4-chloro-7-nitrobenzofurazan, 4-dimethyaminobenzaldehyde, 5,6-dimethylbenzimidazole, 5-chloro-2-methylbenzothiazole, chrysene, 4-hydroxybenzaldehyde, nicotinamide, and camphor;
anthranilic acid, 1,4-naphthoquinone, benzanthrone, tetracene, pentacene, 2-(2-hydroxy-4-methylphenyl)-4-(3)-quinazolone, 2-(3,5-dichloro-2-hydroxyphenyl)-4-(3)-quinazolone, 2-(5-chloro-2-hydroxyphenyl)-4-(3)-quinazolone, 2-(2-hydroxy-3-methylphenyl)-4-(3)-quinazolone, 2-(4-ethyl-2-hydroxyphenyl)-4-(3)-quinazolone, anthraquinone, 1,4-benzoquinone, and salicylic acid; and
thiazoles, liposomes (e.g. Nile Red), stilbenes, azos, azolines, and conjugated polycyclic aromatic compounds containing at least three fused rings that include without limitation anthracene, benzanthracene, pentacene, substituted pentacene, naphthacene, phenacene, substituted phenacene, and derivatives thereof.

9. The developer compound of claim 7 wherein the fluorogenic reagent is selected from one or more of the following reagents:
4-(dimethylamino)cinnamaldehyde,
8-hydroxyquinoline,
Nile Red, Coumarin 1, MBD (7-mehtoxybenzylamino-4-nitrobenzoxadiazole), carbazole, rhodamines,
Dansyl chloride, fluorescamine,
2-(2-hydroxyphenyl)benzoxazole and other low molecular weight benzoxazoles—non-specific Coumarin,
2-)2-hydroxyphenyl)benzothiazole and other low molecular weight benzothiazoles.

10. The developer compound of claim 7 wherein the solvent is selected from one or more of the following:
ehtyl lactate, ethyl acetate, acetone, gamma-butyrolactone, epsilon-caprolactone, 2-phenoxyethanol, carbitols, cellosolves, dichlorobenzene, ethyl benzene, methylene chloride, toluene, o-zylenes, and m-pyrrol.

11. The developer compound of claim 10 wherein the CA is ethyl cyanoacrylate, the fluorogenic reagent is 2-(2-hydroxyphenyl)benzothiazole, and the solvent is ethyl lactate.

12. The developer compound of claim 7 wherein the quantities of CA and the fluorogenic reagent are within the range of about 30 to 60 parts of CA to 1-4 parts of fluorogenic reagent by weight with the solvent comprising the remainder.

13. The developer compound of claim 7 wherein the CA is ethyl cyanoacrylates and the fluorogenic reagent is 2-(2 hydroxyphenyl) benzothiazole and the solvent is ethyl lactate.

14. A method of developing latent fingerprints deposited on a substrate comprising:
a) providing a fluorogenic reagent which will sublimate at a given temperature and is capable of reacting with one or more of the constituents found in the residue of a latent fingerprint to provide a discernable fluorescent image of the fingerprint when subjected to external radiation;
b) mixing the fluorogenic reagent with CA;
c) exposing the substrate or a portion thereof bearing the latent fingerprint to fumes of the fluorogenic reagent CA mixture; and
d) exposing the resulting latent print to said radiation.

15. The method of claim 14 wherein the exposing step includes heating the fluorogenic reagent CA mixture.

16. The method of claim 14 wherein the fluorogenic reagent is one or more of the following reagents:
acridine and its derivatives, rhodamine dyes, 2-(2-hydroxyphenyl)-benzoxazole, 2-(2-hydroxyphenyl)benzothiazole, coumarin compounds such as 4-hydroxycoumarin and coumarins 1, 2,4,6,7,30,102,120,138,151,152,153,307,314, 334,337,338, and 343, benzotriazole, 2-chloro-mercaptobenzoxazole, 2-phenylbenzothiazole, 2-phenylbenzaxazole, 8-hydroxyquinoline, 8-hydroxyquinaldine, anthracene and its derivatives, naphthalene and its derivatives, 4-(dimethylamino)cinnamaldehyde, fluorescamine, phthalic dicarboxaldehyde, naphthoquinone-4-sulfonic acid, dansyl chloride and other dansylated compounds, 4-chloro-7-nitrobenzofurazan, 4-dimethyaminobenzaldehyde, 5,6-dimethylbenzimidazole, 5-chloro-2-methylbenzothiazole, chrysene, 4-hydroxybenzaldehyde, nicotinamide, and camphor;
anthranilic acid, 1,4-naphthoquinone, benzanthrone, tetracene, pentacene, 2-(2-hydroxy-4-methylphenyl)-4-(3)-quinazolone, 2-(3,5-dichloro-2-hydroxyphenyl)-4-(3)-quinazolone, 2-(5-chloro-2-hydroxyphenyl)-4-(3)-quinazolone, 2-(2-hydroxy-3-methylphenyl)-4-(3)-quinazolone, 2-(4-ethyl-2-hydroxyphenyl)-4-(3)-quinazolone, anthraquinone, 1,4-benzoquinone, and salicylic acid; and
thiazoles, liposomes (e.g. Nile Red), stilbenes, azos, azolines, and conjugated polycyclic aromatic compounds containing at least three fused rings that include without limitation anthracene, benzanthracene, pentacene, substituted pentacene, naphthacene, phenacene, substituted phenacene, and derivatives thereof.

17. The method of claim 14 wherein the fluorogenic reagent is one or more of the following reagents:
4-(dimethylamino)cinnamaldehyde,
8-hydroxyquinoline,
Nile Red, Coumarin 1, MBD (7-mehtoxybenzylamino-4-nitrobenzoxadiazole), carbazole, rhodamines,
Dansyl chloride, fluorescamine,
2-(2-hydroxyphenyl)benzoxazole and other low molecular weight benzoxazoles—non-specific Coumarin,
2-)2-hydroxyphenyl)benzothiazole and other low molecular weight benzothiazoles.

18. The method of claim 14 wherein the fluorogenic reagent is 2-(2-hydroxyphenyl)benzothiazole.

19. The method of claim 14 wherein the fluorogenic reagent is Spectrafluor CO Yellow 6 (a roumarin dye-stuff distributed by Spectra Colors Corp.).

20. The method of claim 14 wherein the mixing step includes providing a solvent selected from one or more of the following solvents:
ehtyl lactate, ethyl acetate, acetone, gamma-butyrolactone, epsilon-caprolactone, 2-phenoxyethanol, carbitols, cellosolves, dichlorobenzene, ethyl benzene, methylene chloride, toluene, o-zylenes, and m-pyrrol.
